(19) United States
(12) Patent Application Publication
(10) Pub. No.: US 2015/0012025 A1
(43) Pub. Date: Jan. 8, 2015

(54) AUTOMATIC HANDLE ASSEMBLY FOR SCISSORS-SHEARING

(71) Applicant: HEALTHBANKS BIOTECH CO., LTD., Taipei (TW)

(72) Inventors: Wei-Yu LO, Taipei (TW); Pei-Jan SHEN, Taipei (TW); Yih-Lin CHENG, Taipei (TW); Yen-Ting LEE, Taipei (TW)

(73) Assignee: HEALTHBANKS BIOTECH CO., LTD., Taipei (TW)

(21) Appl. No.: 14/143,278

(22) Filed: Dec. 30, 2013

(30) Foreign Application Priority Data
Jul. 3, 2013 (TW) 102212505

(51) Int. Cl. A61B 17/3201

(52) U.S. Cl. CPC A61B 17/3201; A61B 2017/00398
USPC 606/174

(57) ABSTRACT

An automatic handle assembly for scissors-shearing is a handy device for fixing a scissors to cut tissue, which comprises: a fixture portion including: a fix scissor handle having a proximal end and a distal end, the proximal end equipped with a fix tenon and the distal end equipped with a connection portion; a free scissor handle having a distal end and a proximal end coupled to the fix tenon causing the distal end of the free scissor handle and the distal end of the fix scissor handle to pivot to close or open; and a pair of fixture elements being mounted on the fix scissor handle and the free scissor handle, and slidably moving along with the fix scissor handle and the free scissor handle for adjustably fixing a scissors; a drive unit coupled to the fixture portion to provide reciprocating motion of the free scissor handle to the fix scissor handle; and a power unit being removably equipped on the distal end of the fix scissor handle, and coupled to the drive unit to provide power for the reciprocating motion.

### CROSS REFERENCE

The application claims priority of Taiwan Patent Application NO. 102212505, filed on Jul. 3, 2013, the content thereof is incorporated by reference herein.

### BACKGROUND OF THE INVENTION

1. Field of the Invention

The present invention is related to a fixture assembly, and more particularly to an automatic handle assembly for scissors-shearing.

2. Description of Related Art

Scissors are hand-operated cutting instruments. They are used for cutting various thin materials, such as paper, cardboard, metal foil, thin plastic, cloth, rope, and wire. According to the characteristics of the object, the shearing process will bring some degree of difficulty to the operator. If the object's material is something unregular, the work of cutting process will spend a lot of time.

In Comparison with the general objects, biological specimen cutting should takes more considerations because of the orgams or tissues of human body are unique and special, such as bone, heart, blood vessels, cornea, umbilical cord, and so on.

An umbilical cord that is going to be stored must first be cut from a newborn infant. Ideally, this should be done as soon as possible, and then the cord should be stored in a sterile container before being transported to a lab. Sometimes the whole umbilical cord is stored, but other times just a piece of the cord is stored. The umbilical cord is easily obtained after the birth of the newborn, is normally thrown away and poses no risk for collection. The umbilical cord mesenchymal stem cells (MSCs) have more primitive properties than other adult MSCs obtained later in life, which might make them a useful source of MSCs for clinical applications. An extremely rich source for mesenchymal stem cells is the developing tooth bud of the mandibular third molar. While considered multipotent, they may prove to be pluripotent. The stem cells eventually form enamel, dentin, blood vessels, dental pulp, nervous tissues, including a minimum of 29 different unique end organs. Umbilical cord tissue banking is beneficial because the tissue contains a rich source of MSCs, which are increasingly being utilized in regenerative medicine research, targeting potential therapies for a wide range of conditions including heart disease, stroke, multiple sclerosis and diabetes.

Heretofore, since general medical scissors made of linear blade of scissors has been used in maternity clinic in hospital, in case of cutting the umbilical cord, the umbilical cord was put in the culture dish whereby operator cut the umbilical cord into small pieces for cells culture. But the cutting of umbilical cord was not easy, and cutting of correct portion of umbilical cord to be cut off was also difficult, and since the umbilical cord is a somewhat tough, gelatinous, fibrous tissue member having two arteries and a vein, and the cord is usually covered with at least amniotic fluid and blood upon delivery. The diameter of the cord varies from about one-quarter to one inch, with greater force required to secure the clamps onto the thicker cords. So 2.5 hours to 3.5 hours was needed to cut just 5 cm umbilical cord, this process required long standing posture and hand-working to obtain useful output of cell culture, the operator tend to have occupational injury, like Carpal tunnel syndrome.

Further, an umbilical cord clipping machine was made, which was similar to blender or meat mincer type device, to obtain tiny pieces of umbilical cord. The machine seems saving manpower and time to prepare the cells in the culture dish, and cutting same small size of tissue. But the cell culture process needs biological cell, not the damaged cells which is cut continuously and repeatedly by the blades in the machine. The disadvantage of the cord clipping machine is unable to obtain cell recovery rate as good as hand-cut tissue for generating the Mesenchymal Stem Cell. therefore there has been also disadvantage that the portion of umbilical cord to be cut was hard to be correctly cut off.

And, in order to prevent that the umbilical cord slips along toward tip end direction of the scissors in case of using the general medical scissors made of linear blade of scissors upon cutting the umbilical cord by operating person, a scissors that both blades of scissors were bent in arcuate form to innerward has been developed, however such scissors also splashes the blood around to every direction upon cutting the umbilical cord by operating person whereby becoming to smear the hand, face, and operating gown of operating person as well as sheet of bed, therefore there has been various disadvantages that it is uneconomical and having a worry about infection of disease and the like.

In view of the foregoing, a need exists in the art for a system and method for measuring surface flatness of continuously moving samples. In addition, a need exists for such a system and method to be efficient and cost-effective.

### SUMMARY OF THE INVENTION

In general, an automatic handle assembly for scissors-shearing is provided having various aspects addressed to overcome the shortcomings in prior scissor instruments discussed above and provide certain other advantages.

The primary objective of the present invention is to provide an automatic handle assembly for scissors-shearing, which is a handy device for fixing a scissors to cut tissue in replacement of hand-cut operation.

The next objective of the present invention is to provide an automatic handle assembly for scissors-shearing to increase the process of umbilical cord cutting, for improving the efficiency of operation and saving cutting time.

The another objective of the present invention is to provide an automatic handle assembly for scissors-shearing to replace hand cutting work of umbilical cord for preventing the injury of personal's hand.

In order to accomplish the above objective, the automatic handle assembly for scissors-shearing is used to fix a shearing device, which comprises: a fixture portion including: a fix scissor handle having a proximal end and a distal end, the proximal end equipped with a fix tenon and the distal end equipped with a connection portion; a free scissor handle having a distal end and a proximal end coupled to the fix tenon causing the distal end of the free scissor handle and the distal end of the fix scissor handle to pivot to close or open; and a pair of fixture elements mounted on the fix scissor handle and the free scissor handle, and slidably moving along with the fix scissor handle and the free scissor handle for adjustably fixing a scissors; a drive unit coupled to the fixture portion to provide reciprocating motion of the free scissor handle to the fix scissor handle; and a power unit removably equipped on the distal end of the fix scissor handle, to the drive unit to provide power for the reciprocating motion.

The invention of automatic handle assembly has a fixture portion to fix the blades of a scissors for cutting tissue, for instance the umbilical cord. The operator combines the medical umbilical scissors with fixture portion, then the drive unit provides reciprocating motion of the free scissor handle to the fix scissor handle for sheering motion of the scissors.

Therefore, there are three fixing points on the automatic handle assembly for adjustably holding the medical scissors, thus two devices work as one auto apparatus. The power unit is coupled to the drive unit to provide power for the reciprocating motion of the free scissor handle to the fix scissor handle for shearing.

The assembly steps includes: fixture elements slidably moving along with the fix scissor handle and the free scissor handle to fix the blades of medical scissors; the pivot of the medical scissors positioned on a fix tenon of the fix scissor handle; and two fixture elements fixing the finger loops respectively.

Furthermore, the invention of automatic handle assembly for scissors-shearing not only can replace the hand-cut operation to promote the cutting process, but also reduce shearing time and the chance of injured hands. Moreover, the effectiveness of culturing the cells from the umbilical cord tissue cut by the invention is as good as the traditional hand cut operation. In comparison with umbilical cord clipping machine, which is similar to blender or meat mincer type device, the invention prevents damaging the cells in order to achieve reproducible results from cells.

Therefore, the design of the automatic handle assembly is non-obvious and sufficiently inventive, and reflects a same general patentability requirement. It is noted that the present invention has the advantages that the invention provides a clear and ergonomic instrument for medical personnel concerning the operation of umbilical cord clipping and making practical progress on the field.

### BRIEF DESCRIPTION OF THE DRAWINGS

An embodiment of the invention will now be described, by way of example only, with reference to the accompanying drawings in which:

FIG. 1 is an isometric view of the automatic handle assembly of the preferred embodiment in accordance with the present invention;

FIG. 2 is another isometric view of the automatic handle assembly of the preferred embodiment in accordance with the present invention;

FIG. 3 is an assembled view of the automatic handle assembly of the preferred embodiment in accordance with the present invention;

FIG. 4 is another assembled view of the automatic handle assembly of the preferred embodiment in accordance with the present invention;

FIG. 5 is an assembled view of the fixture portion of the preferred embodiment in accordance with the present invention;

FIG. 6 is another assembled view of the fixture portion of the preferred embodiment in accordance with the present invention;

FIG. 7 is an assembled view of the fix scissor handle and the free scissor handle of the preferred embodiment in accordance with the present invention;

FIG. 8 is an assembled view of the fix scissor handle, the free scissor handle and the first gear of the preferred embodiment in accordance with the present invention;

FIG. 9 is a perspective view of the fixture elements slidably positioned on the fix scissor handle and the free scissor handle of the preferred embodiment in accordance with the present invention;

FIG. 10 is a perspective view of the handle assembly fixing a scissors in accordance with the present invention;

FIG. 11 is a perspective view of the handle assembly and the scissors in accordance with the present invention;

FIG. 12 is a shearing view in accordance with the present invention;

FIG. 13 is another shearing view in accordance with the present invention;

FIG. 14 is still another shearing view in accordance with the present invention;

FIG. 15A is a picture showing primary culture of umbilical cord through 10 days observation cut by the invention in accordance with the present invention;

FIG. 15B is a picture showing primary culture of umbilical cord through 10 days observation cut by a scissors in prior art;

FIG. 16A is a picture showing the result of subculture to P1 in accordance with the present invention; and

FIG. 16B is a picture showing primary culture of umbilical cord through 10 days observation cut by a scissors in prior art.

### DETAILED DESCRIPTION OF THE INVENTION

The following description is merely exemplary in nature and is in no way intended to limit the present teachings, applications, or uses. Those of skill in the art will recognize that the following description is merely illustrative of the principles of the invention, which may be applied in various ways to provide many different alternative embodiments.

The invention relates to an automatic handle assembly for scissors-shearing, which holds and moves a specific surgical scissors to cut baby's umbilical cord to prevent and even cure a wide range of life-threatening diseases in his/her continuous life.

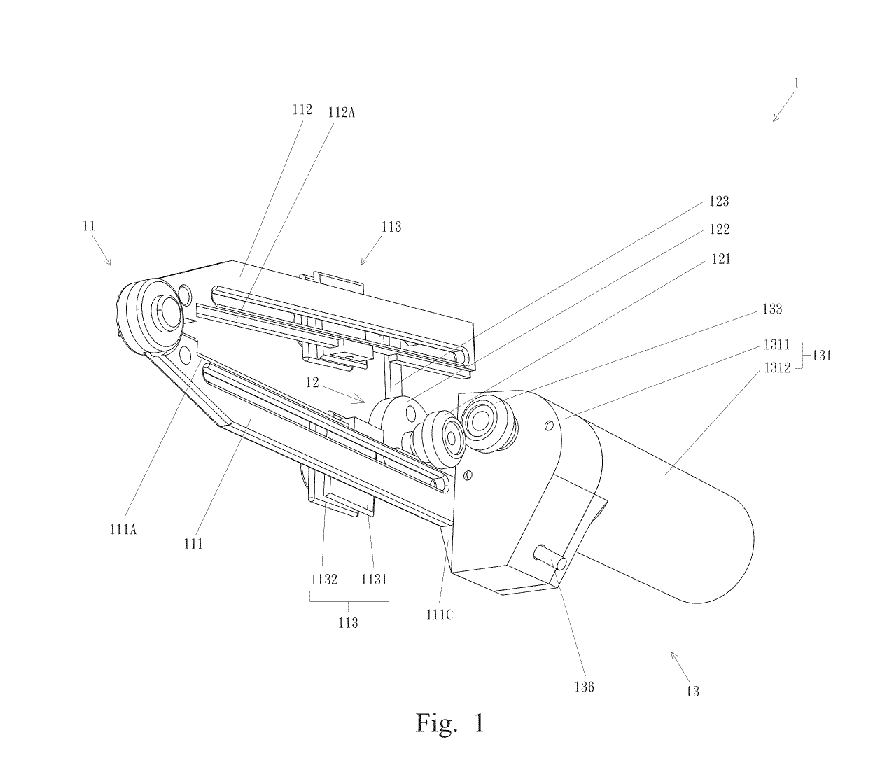

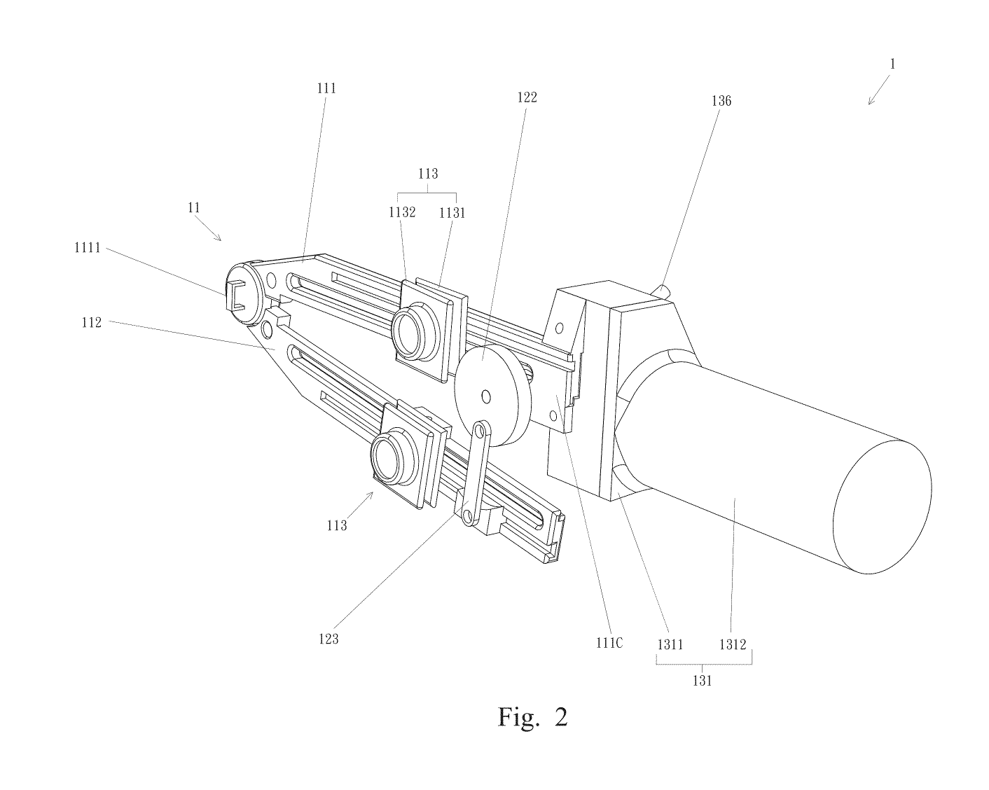

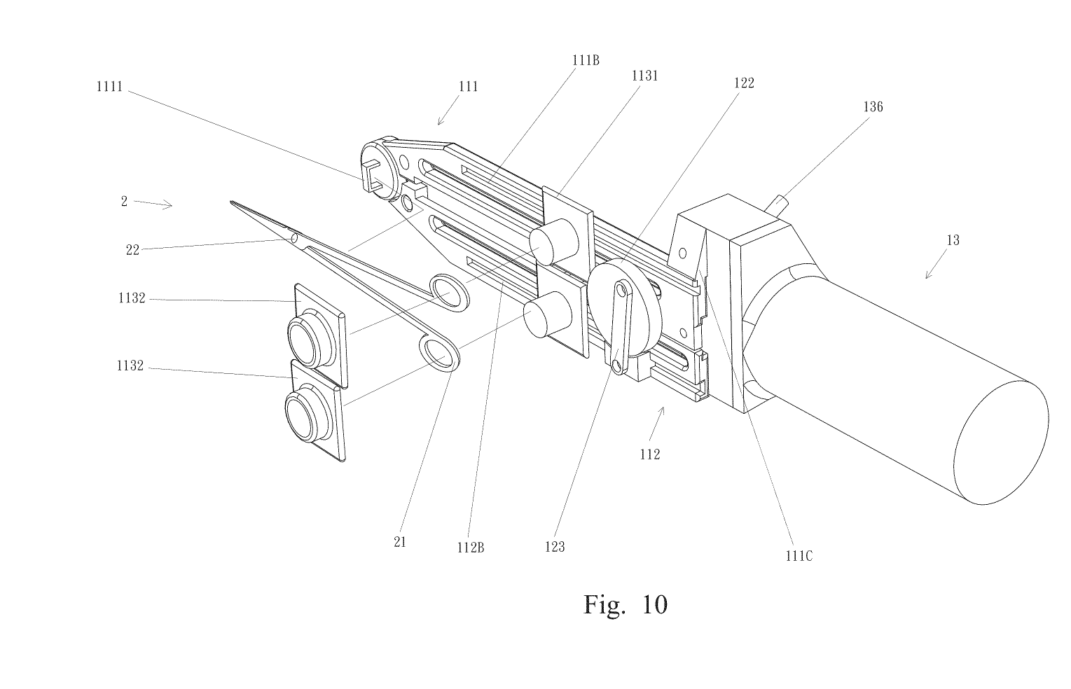

FIGS. 1, 2 and 10 are illustrations of the preferred embodiment in accordance with the present invention. There is shown the automatic handle assembly 1 includes a fixture portion 11, a driver unit 12 and a power unit 13.

A fixture portion 11 includes a fix scissor handle 111 and a free scissor handle 112 to hold two arms of the surgical scissors 2. The fix scissor handle 111 has a proximal end and a distal end, the proximal end equipped with a fix tenon 1111 and the distal end equipped with a connection portion 111C. The free scissor handle 112 has a distal end and a proximal end coupled to the fix tenon 1111 causing the distal end of the free scissor handle 112 and the distal end of the fix scissor handle 111 to be pivoted to close or open.

Pair of fixture elements 113 in the fixture portion 11 are mounted on the fix scissor handle 111 and the free scissor handle 112, and slidably moving along with the fix scissor handle 111 and the free scissor handle 112 for adjustably fixing the surgical scissors.

The drive unit 12 is coupled to the fixture portion 11 to provide reciprocating motion of the free scissor handle to the fix scissor handle. The power unit 13 is removably equipped on the distal end of the fix scissor handle 111, and coupled to the drive unit 12 to provide power for the reciprocating motion.

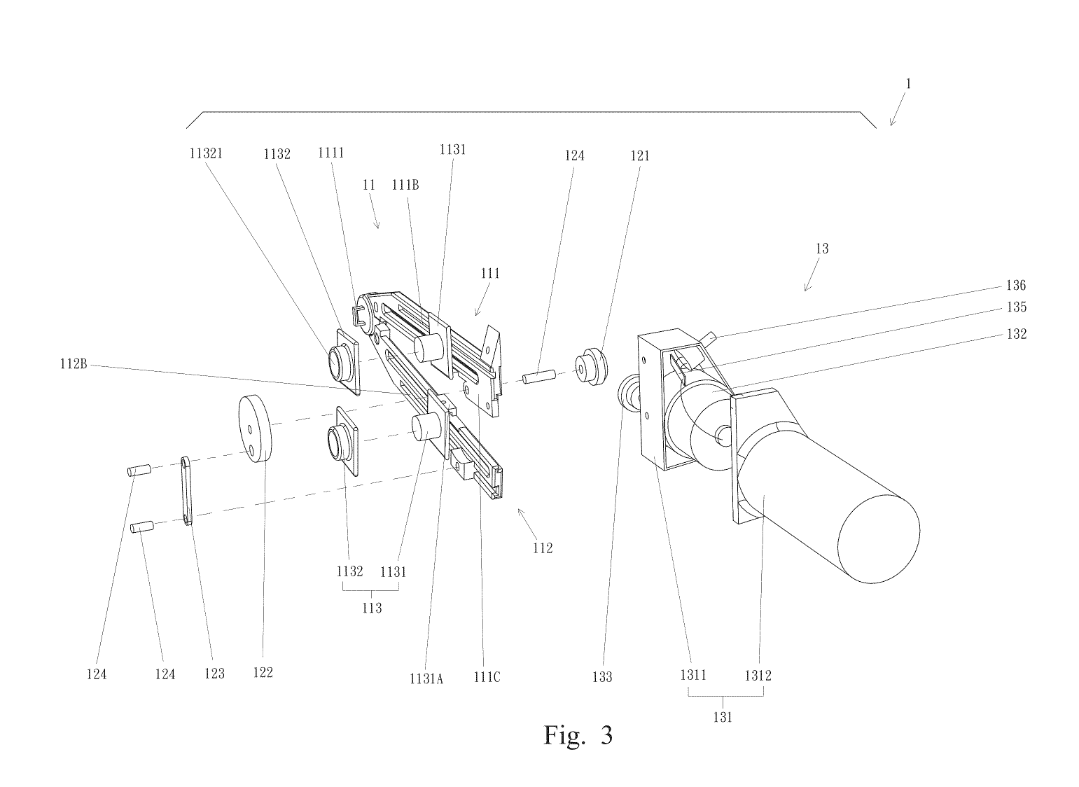

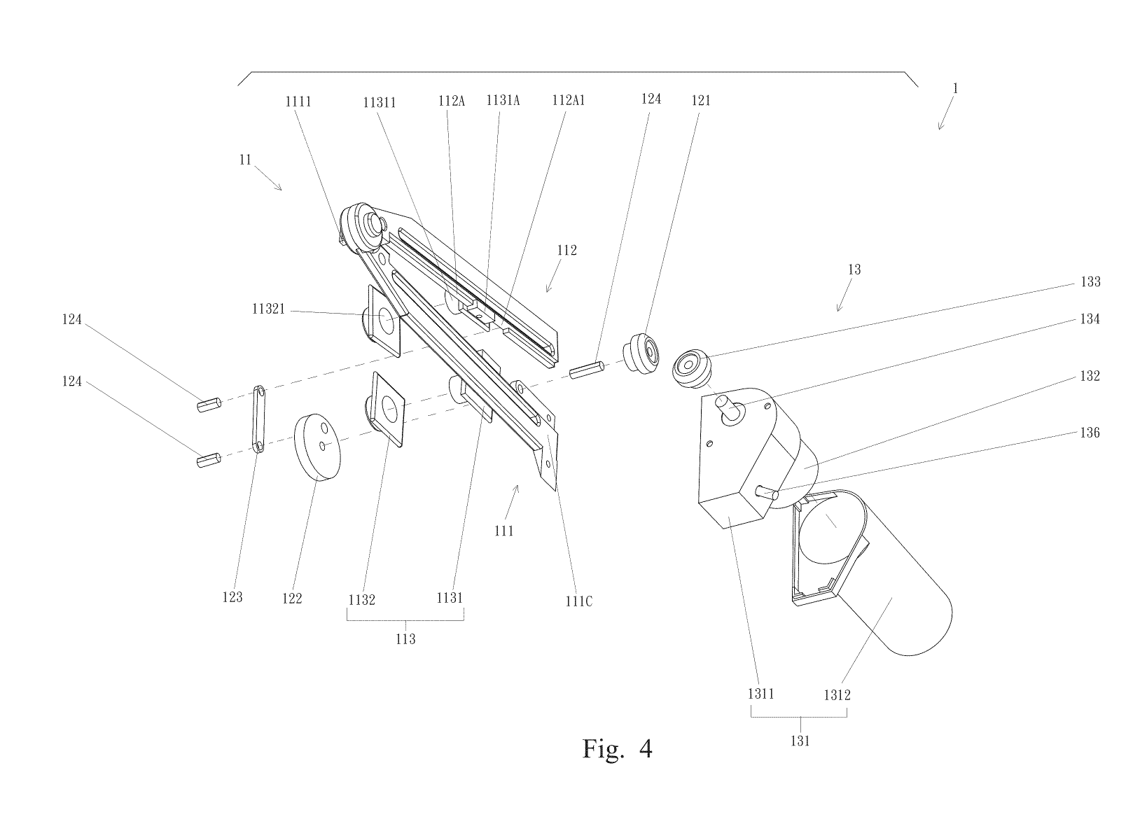

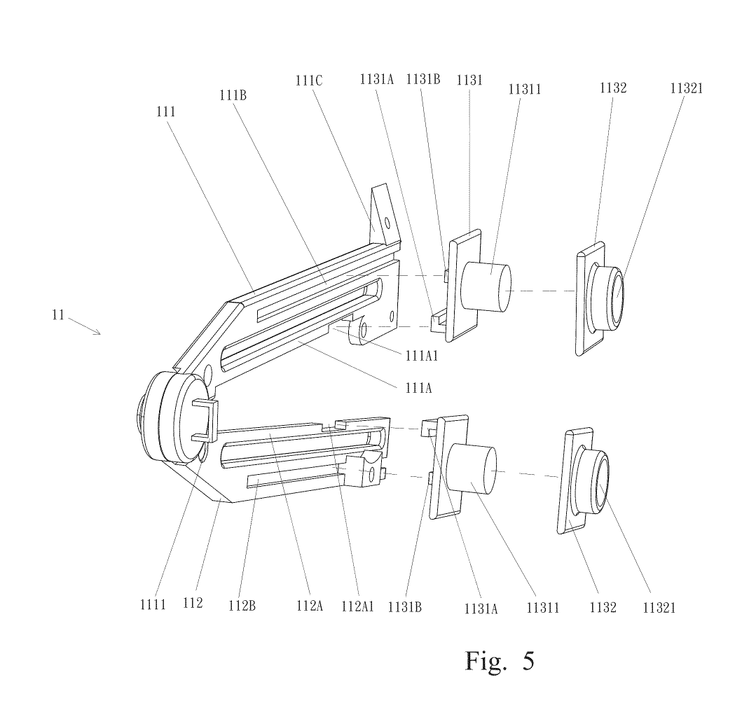

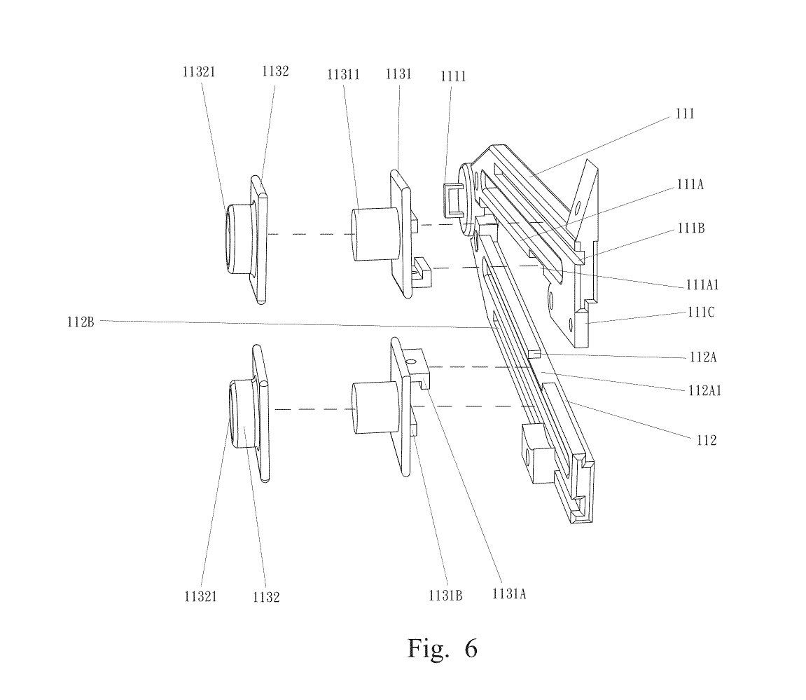

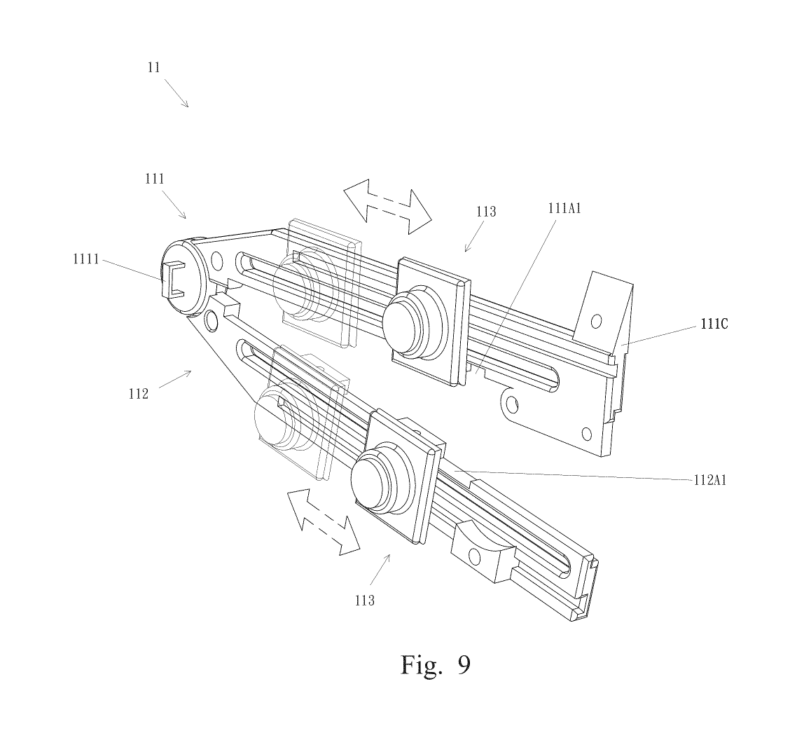

FIGS. 3-6, 9 and 10 are illustrations of the preferred embodiment in accordance with the present invention that the automatic handle assembly is illustrated in explosive views and assembled views. The pair of fixture elements 113 includes a first fixture element 1131 and a second fixture element 1132 positioned on the fix scissor handle 111 and free scissor handle 112 respectively to fix two arms of the surgical scissors respectively. The screw lock or buckling way may be used to fix two arms of the surgical scissors 2. In this embodiment the buckling way shows that screw rods 11311 are positioned on one side of the first fixture element 1131 to put through to one of finger loops on the surgical scissors 2. Nuts 11321 are used to restrain the blades of the surgical scissors 2 with the screw rods 11311, so the surgical scissors 2 is fixed between the first fixture element 1131 and the second fixture element 1132. In the embodiment, the outer surface of the screw rod 11311 and the inner surface of the nut 11321 are formed having complementary helical threads which are generally semi-circular in cross section.

Each of the fix scissor handle 111 and the free scissor handle 112 has at least one slide track along with the axis for the fixture elements 113 moving along the slide tracks. The slide tracks disposed on the inner face of the fix scissor handle 111 and the free scissor handle 112 are first slide tracks 111A, 112A. The second slide tracks 111B, 112B are laterally disposed on the fix scissor handle 111 and the free scissor handle 112 with respect to the first slide tracks 111A, 112A. The fix scissor handle 111 and the free scissor handle 112 have two openings 111A1, 112A1 on the first slide tracks 111A, 112A.

The fixture element 113 has a plurality of slide guides corresponding to the slide tracks in the first fixture element 1131. In the embodiment the first slide guides 1131A are mounted on the first slide tracks 111A, 112A from the opening 111A1, 112A1. The second slide guides 1131B are fixed into the first slide tracks 111B, 112B.

The first slide guides 1131A are positioned on the opening 111A1, 112A1, and then the second slide guides 1131A are faced to the second slide tracks 111B, 112B. Pushing the first slide guides 1131A into the first slide tracks 111A, 112A, the first fixture element 1131 is combined to the fix scissor handle 111 and the free scissor handle 112. By the slide guides equipped with the slide tracks, the first fixture elements 1131 are in the fix scissor handle 111 and the free scissor handle 112 for slidably moving along with the slide tracks. The mechanism of slide and track makes most of surgical scissors with loop rings fixed on the fix scissor handle 111 and the free scissor handle 112.

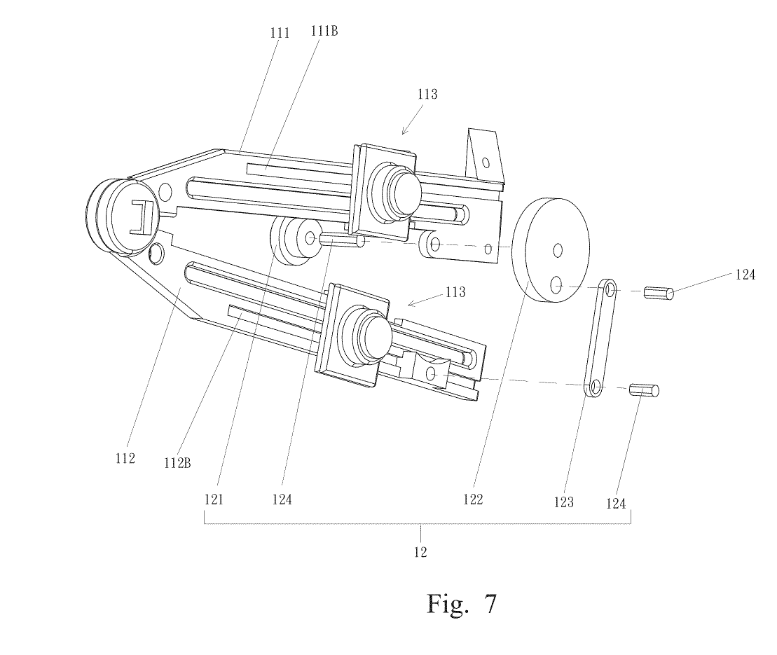

Referring to FIGS. 1, 3, 4 and 7, the drive unit 12 includes a first gear equipped on the connect portion, a rotating disk equipped on the connection portion and rotatably joined with the first gear, and an actuation rod connecting the free handle and the rotating disk with two ends to translate motion from the first gear to the free handle.

The drive unit 12 has a hinge pin 124 disposed through the connection portion 111C to connect the first gear 121 and the rotating disk 122 for transmitting rotation movement. There are two hinge pins 124′ of cylindrical metals that holds together the fix scissor handle and the free scissor handle with an actuation rod 123 in a manner that the two handles can freely close or open.

The invention may use any drive unit to provide reciprocating motion of the free scissor handle to the fix scissor handle; the illustration is just one of clear embodiments.

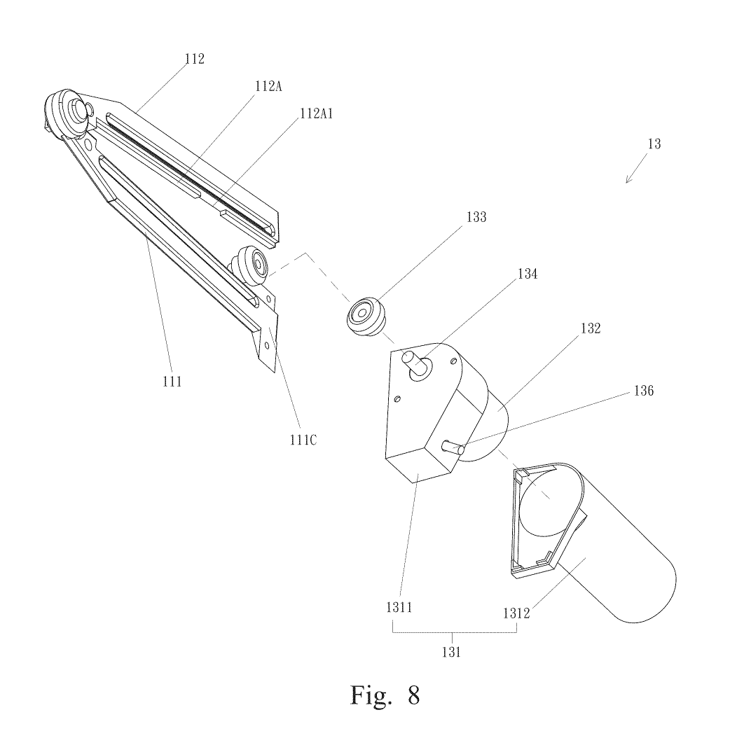

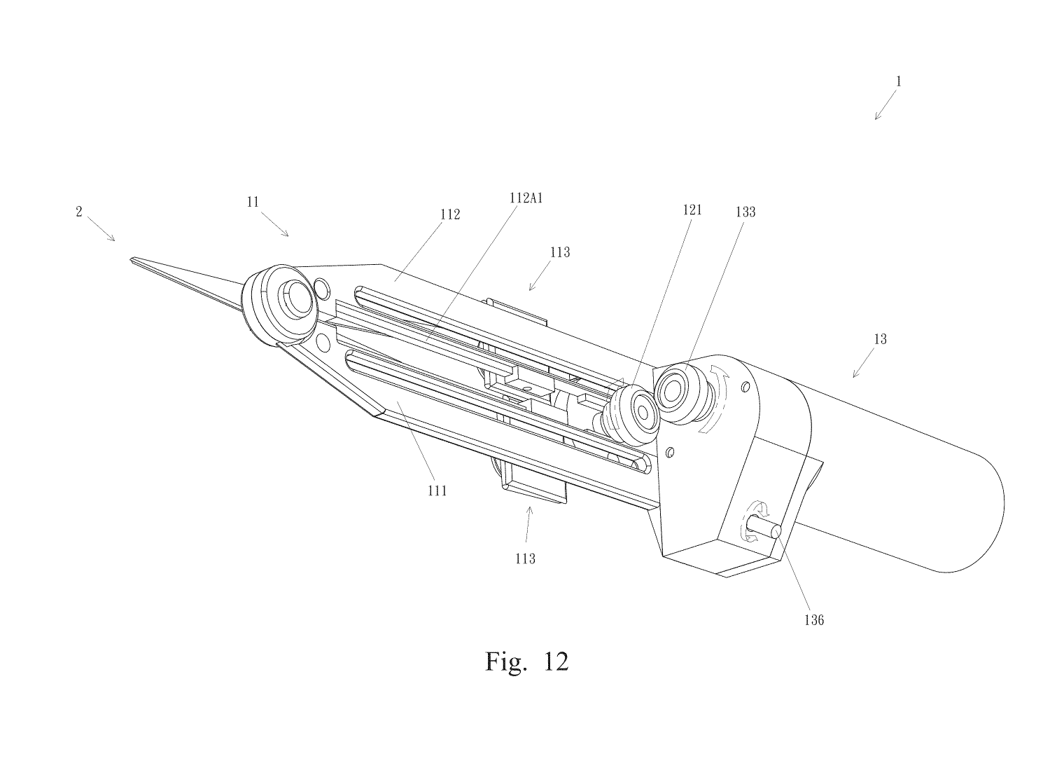

Referring to FIGS. 1, 3, 4, 8 and 12, the power unit 132 includes a hollow extended grip 131 extended from the connect portion 111C, a mechanic power unit 132 equipped inside the hollow extended grip 131, and a second gear 133 driven by the mechanic power unit to engage the first gear 121. By engaging the first gear 121 with the second gear 133, the power unit 13 outputs mechanic energy to the drive unit 12.

Accordingly, the hollow extended grip 131 has a cover 1311 equipped to the connect portion 111C of the fix scissor handle 111, and a holding portion 1312 binding to the cover 1311, which is detachably arrange on the connect portion 111C. The power unit 13 is installed in the fix scissor handle.

The power unit 13 further includes: a drive shaft 134 disposed inside the grip 131 and protruding from the cover 1311 to connect the second gear 133 for transmitting rotation from a mechanic power unit 132; a control electric circuit having a speed regulator 135; and a variable resister 136 electrically coupled to the speed regulator 135 to adjust the speed of the mechanic power unit 132 by a user. An operator can control the driver unit 12 by using the variable resister 136 to adjust power unit 13 through the speed regulator 135, that the driver unit 12 is controlled by the speed regulator 135 through the power unit 13, which provides reciprocating motion of the free scissor handle 112 to the fix scissor handle 111.

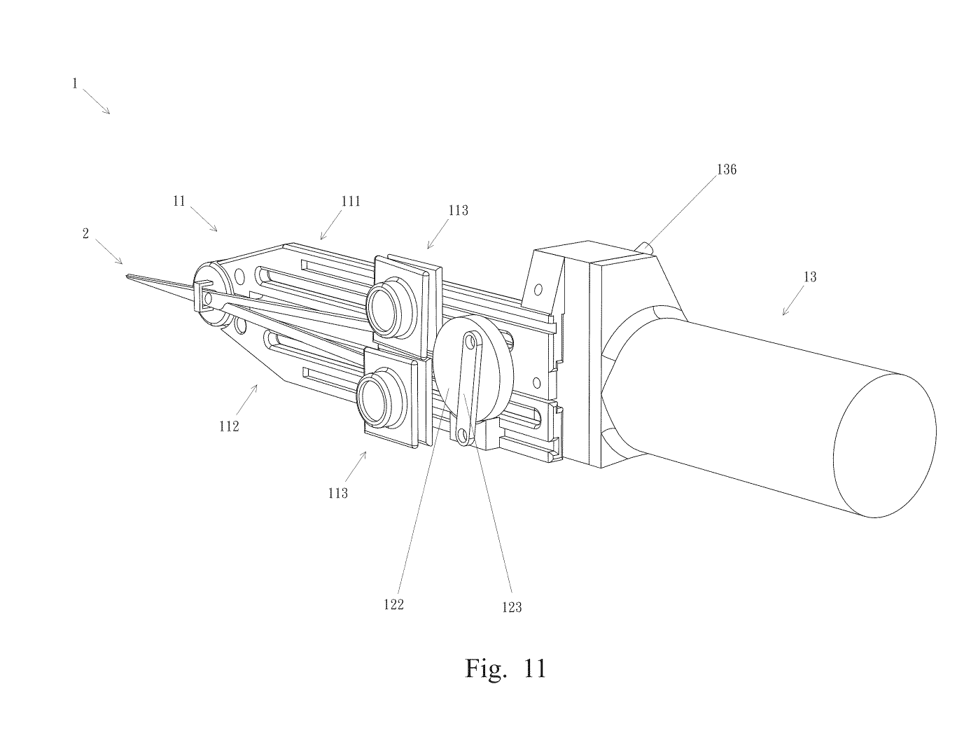

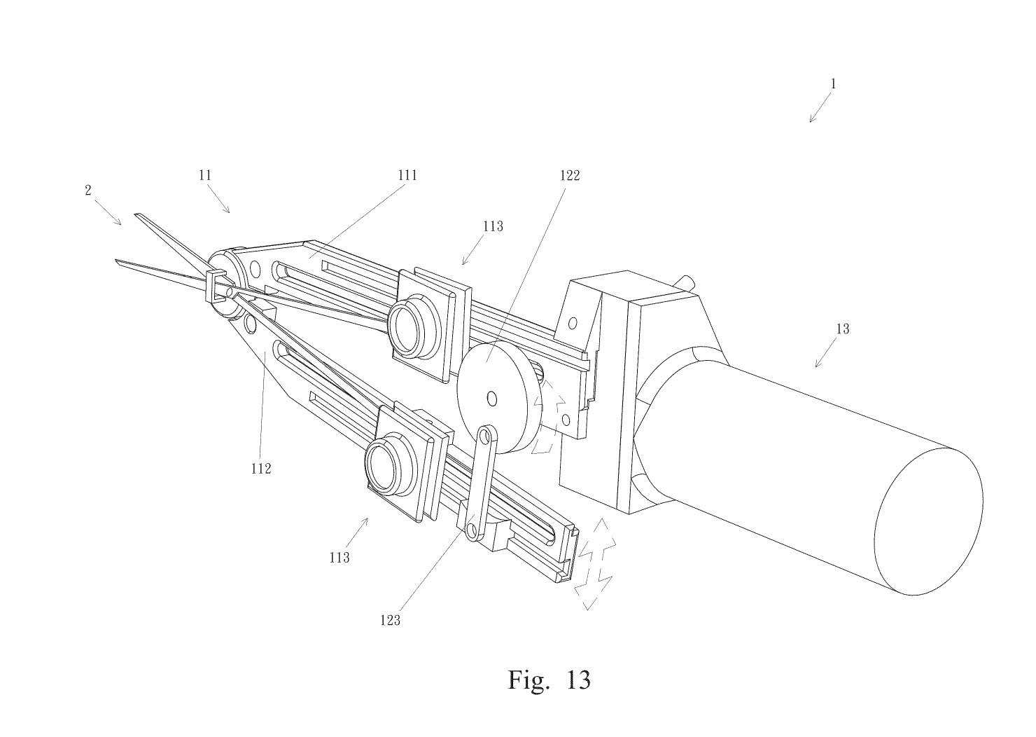

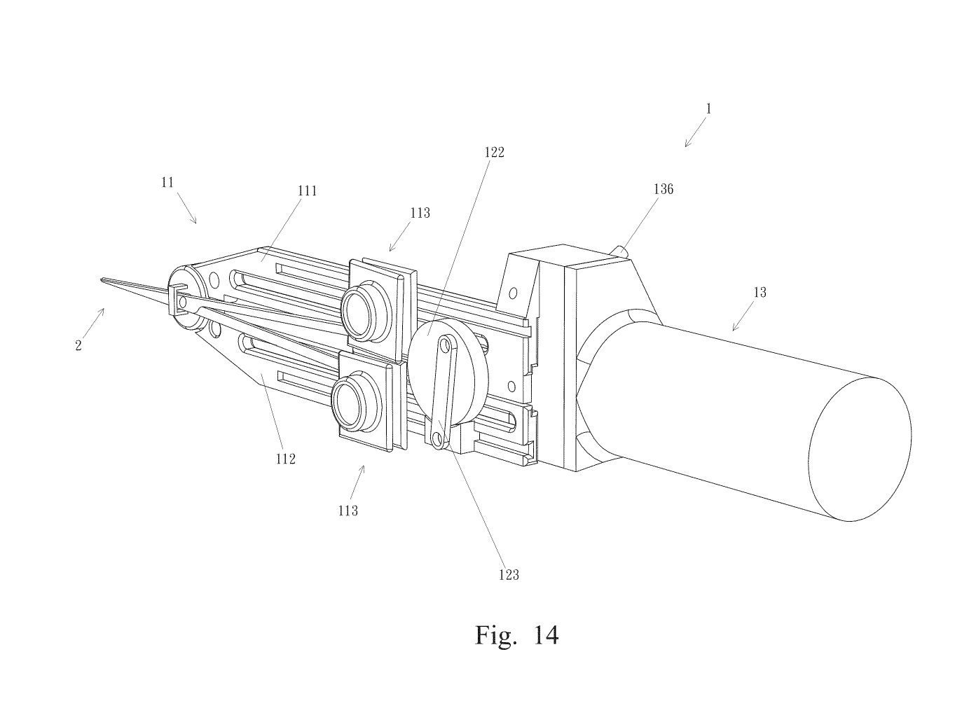

Referring to FIGS. 3 and 8-14, in the operation of umbilical cord cutting, operator uses the embodiment of automatic handle assembly 1 to control a surgical scissors 2. The fixture elements 113 are adjusted to let the finger loops 21 hookup the screw rods 11311 of the first fixture element. The pivot 22 of the surgical scissors 2 is fixed in the fix tenon 1111 of the fix scissor handle 111, then the second fixture elements 1132 screw to the screw rods 11311 to lock the blades of the surgical scissors 2.

Accordingly, the operator starts up the power unit 132 by turning a variable resistor to path the electric current. A drive shaft, which disposed inside the grip 131 and protrudes from the cover 1311, is connected to the second gear 133 for transmitting rotation from an electric motor (not shown). The rotating of the second gear 133 meshes the first gear 121 in order to transmit torque to the rotating disk 122. Then, the rotating disk 122 having same axis with the second gear 133 rotates one end of the actuation rod 122 in circle to move the free scissor handle 112 close to and away from the fix scissor handle 111. By the drive force, the reciprocating motion of the free scissor handle 112 and the fix scissor handle 111 provides shearing motion to the surgical scissors 2, which has finger loops 21 fixed on the first fixture element 1131 and the second fixture element 1132. The advantage of this design is that the actuation rod 123 and the rotating disk 122 provide a quick return mechanism to control the surgical scissors 2 for instantly cutting the umbilical cord to tiny pieces, whereby the successful cutting of umbilical cord is easy.

Accordingly, the handles of surgical scissors 2 positioned on the automatic handle assembly 1 follows the reciprocating motion of the free scissor handle 112 and the fix scissor handle 111 to cut off the umbilical cord. For cutting the umbilical cord, the operator just hold the grip 131 and position it with right angle to the cord, thereby, the hand working of the cord cutting can be replaced by machine, saving the time and avoiding the pathologies, such as carpal tunnel syndrome, nerve injury, tendon injuries of the hand, and neuromuscular disorders.

For one more important purpose of the invention, the efficiency of cell culture of the umbilical cord tissue cut by the surgical scissors with automatic handle assembly should equal to or better than the cord tissue cut by hand operation. For comparing the invention to the hand cutting, three analyses are used to evaluate the efficiency of cell culture, which includes: Observation of cell morphology, Analysis of cell number P₀ and Analysis of cell proliferation.

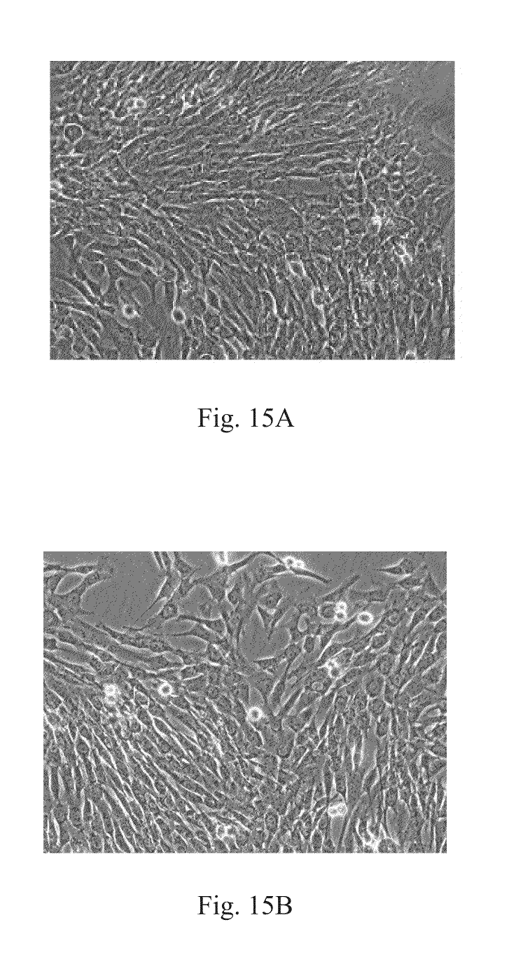

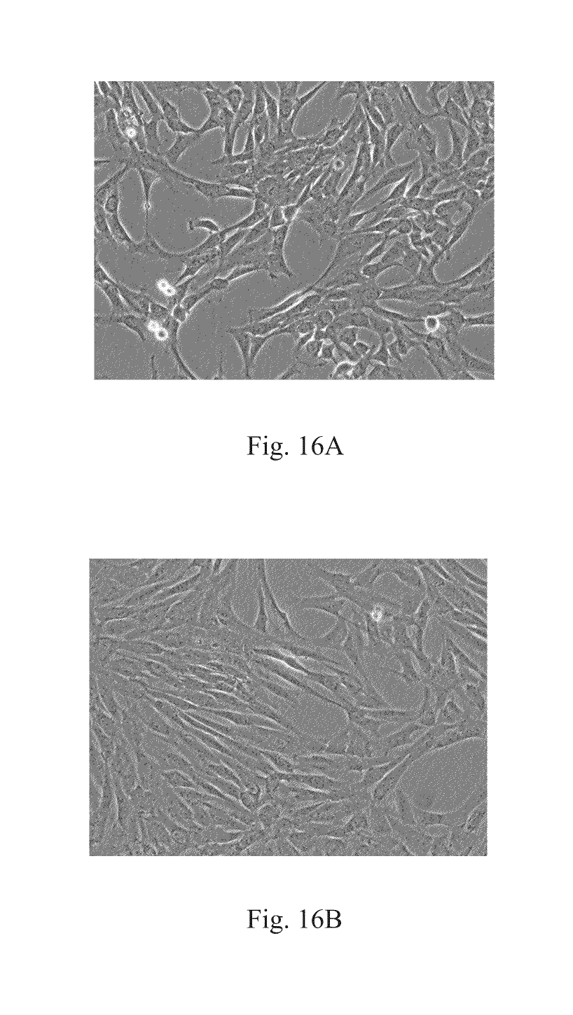

Referring to FIGS. 15A, 15B, 16A and 16B, the observation of cell type are magnified 100 times under the microscope. FIGS. 15A and 15B show primary culture of umbilical cord through 10 days observation, and FIGS. 16A and 16B show the result of subculture to P1.

Accordingly, the umbilical cord tissue cut by the invention is active, some cells can be found out of tissue block after 10 days of Primary culture Fibroblast-like cells can be found in both of hand cut and automatic cut. Furthermore, the P0 cells from both sides are continued to go P1 subculture, both group of cells are Fibroblast-like. There is no difference between hand-cut tissue and automatic cut tissue.

Table 1 shows that two groups of cells obtained from different cutting. The invention has only 2 more days than hand-cut process at primary culture, but produces more P0 cells than hand-cut process.

TABLE 1
The amount of cells after the primary culture from the umbilical
cord tissue.

| Group | Primary culture days | Amount of cells |
|---|---|---|
| Hand-cut | 10 days | 6.67 * 105 |
| The invention | 12 days | 16.20 * 105 |

Table 2 shows that the rate of speed obtained from two groups of cell. The invention has less about 40 minutes than hand-cut process at P0 to P1 culture.

TABLE 2
The amount of cells after the primary culture from the umbilical
cord tissue.

| Group | Primary culture days |
|---|---|
| Hand-cut | 10 days |
| The invention | 12 days |

Through the observation and analysis of cell morphology, cell number, proliferation time, tissue cut by the invention provides good efficiency of umbilical cord tissue cells as the hand cut tissue.

The embodiment of the present invention is the material of choice, it is recommended that the preferred ordinary steel, followed by stainless steel, special requirements can be used in carbon steel and heat treatment is carried out.

From the above description, the invention of automatic handle assembly for scissors-shearing not only can replace the hand-cut operation to promote the cutting process, but also reduce shearing time and the chance of wounded hands. Moreover, the effectiveness of culturing the cells from the umbilical cord tissue cut by the invention is as good as the traditional hand cut operation. In comparison with umbilical cord clipping machine, which is similar to blender or meat mincer type device, the invention prevents damaging the cells in order to achieve reproducible results from cells.

The invention is non-obvious and sufficiently inventive, and reflects a same general patentability requirement. It is noted that the present invention has the advantages that the invention provides a clear and ergonomic instrument for medical personnel concerning the operation of umbilical cord clipping and making practical progress on the field.

While the invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. An automatic handle assembly for scissors-shearing, which comprising:
a fixture portion including:
a fix scissor handle having a proximal end and a distal end, the proximal end equipped with a fix tenon and the distal end equipped with a connection portion;
a free scissor handle having a distal end and a proximal end coupled to the fix tenon causing the distal end of the free scissor handle and the distal end of the fix scissor handle to pivot to close or open; and
a pair of fixture elements mounted on the fix scissor handle and the free scissor handle, and slidably moving along with the fix scissor handle and the free scissor handle for adjustably fixing a scissors;
a drive unit coupled to the fixture portion to provide reciprocating motion of the free scissor handle to the fix scissor handle; and
a power unit removably mounted on the distal end of the fix scissor handle to the drive unit to provide power for the reciprocating motion.

2. The automatic handle assembly for scissors-shearing as claimed in claim 1, wherein each of the pair of fixture elements includes a first fixture element and a second fixture element positioned on and slidably moving along with the fix scissor handle and free scissor handle respectively to fix two blades of a scissors respectively.

3. The automatic handle assembly for scissors-shearing as claimed in claim 1, wherein each of the fix scissor handle and the free scissor handle has at least one slide track defined along with an axis thereof for the fixture elements to move along the slide track.

4. The automatic handle assembly for scissors-shearing as claimed in claim 2, wherein each of the fix scissor handle and the free scissor handle has at least one slide defined along with axes thereof to allow the fixture elements to move along the slide.

5. The automatic handle assembly for scissors-shearing as claimed in claim 1, wherein the drive unit includes a first gear equipped on the connect portion, a rotating disk equipped on the connection portion and rotatably joined with the first gear, and an actuation rod connecting the free handle and the rotating disk with two ends to translate motion from the first gear to the free handle.

6. The automatic handle assembly for scissors-shearing as claimed in claim 5, wherein the power unit includes a hollow extended grip extending from the connect portion, a mechanic power unit equipped inside the hollow extended grip, and a second gear driven by the mechanic power unit to engage the first gear.

7. The automatic handle assembly for scissors-shearing as claimed in claim 2, wherein the first fixture element has a screw rod and the second fixture element has a nut corresponding to the screw rod.

8. The automatic handle assembly for scissors-shearing as claimed in claim 3, wherein the slide tracks disposed on the inner face of the fix scissor handle and the free scissor handle are first slide tracks, the slide tracks laterally disposed on the fix scissor handle and the free scissor handle with respect to the first slide tracks are second slide tracks.

9. The automatic handle assembly for scissors-shearing as claimed in claim 4, wherein the slide tracks disposed on the inner face of the fix handle and the free handle are first slide tracks, the slide tracks laterally disposed on the fix handle and the free handle with respect to the first slide tracks are second slide tracks.

10. The automatic handle assembly for scissors-shearing as claimed in claim 4, wherein the first fixture element has at least one slide guide corresponding to the slide track.

11. The automatic handle assembly for scissors-shearing as claimed in claim 9, wherein the first fixture element has at least one slide guide corresponding to the slide track.

12. The automatic handle assembly for scissors-shearing as claimed in claim 11, wherein the slide guides includes a first slide guide formed on the first fixture element corresponding to the first slide track, and a second slide guide formed on the second fixture element corresponding to the second slide track.

13. The automatic handle assembly for scissors-shearing as claimed in claim 6, wherein the hollow extended grip has a cover equipped to the connect portion of the fix scissor handle, and a holding portion binding to the cover, the cover is detachably arranged on the connect portion.

14. The automatic handle assembly for scissors-shearing as claimed in claim 6, wherein the power unit includes:
a drive shaft disposed inside the grip and protruding from the cover to connect to the second gear for transmitting rotation from a electric motor;
a control electric circuit electrically coupled to the electric motor for controlling the electric motor; and
a speed regulator coupled to the electric motor for adjusting speed of the electric motor by a user.

15. An automatic handle assembly for scissors-shearing, the automatic handle assembly comprising:
a fixture portion including:
a fix scissor handle having a proximal end and a distal end, the proximal end equipped with a fix tenon and the distal end equipped with a connection portion;
a free scissor handle having a distal end and a proximal end coupled to the fix tenon causing the distal end of the free scissor handle and the distal end of the fix scissor handle to pivot to close or open; and;
a pair of fixture elements mounted on the fix scissor handle and the free scissor handle, and slidably moving along with the fix scissor handle and the free scissor handle for adjustably fixing a scissors;
a drive unit coupled to the fixture portion to provide reciprocating motion of the free scissor handle to the fix scissor handle, the drive unit including:
a first gear equipped on the connect portion;
a rotating disk equipped on the connection portion and rotatably joined with the first gear; and
an actuation rod connecting the free handle and the rotating disk with two ends to translate motion from the first gear to the free handle; and
a power unit removably mounted on the distal end of the fix scissor handle to the drive unit to provide power for the reciprocating motion, the power unit including:
a hollow extended grip extending from the connect portion;
a mechanic power unit equipped inside the hollow extended grip; and
a second gear driven by the mechanic power unit to engage the first gear.

16. The automatic handle assembly for scissors-shearing as claimed in claim 15, wherein each of the pair of fixture elements includes a first fixture element and a second fixture element positioned on and slidably moving along with the fix scissor handle and free scissor handle respectively to fix two blades of a scissors respectively.

17. The automatic handle assembly for scissors-shearing as claimed in claim 16, wherein the first fixture element has a screw rod and the second fixture element has a nut corresponding to the screw rod.

18. The automatic handle assembly for scissors-shearing as claimed in claim 16, wherein each of the fix scissor handle and the free scissor handle has at least one slide track defined along with an axis thereof for the fixture elements having at least one slide track to move along with.

19. The automatic handle assembly for scissors-shearing as claimed in claim 15, wherein the hollow extended grip has a cover equipped to the connect portion of the fix scissor handle, and a holding portion binding to the cover, the cover is detachably arranged on the connect portion.

20. The automatic handle assembly for scissors-shearing as claimed in claim 15, wherein the power unit includes:
a drive shaft disposed inside the grip and protruding from the cover to connect to the second gear for transmitting rotation from a electric motor;
a control electric circuit electrically coupled to the electric motor for controlling the electric motor; and
a speed regulator coupled to the electric motor for adjusting speed of the electric motor by a user.
